(19) United States
(12) Patent Application Publication
(10) Pub. No.: US 2023/0227809 A1
(43) Pub. Date: Jul. 20, 2023

(54) Multiplex Chromatin Interaction Analysis with Single-Cell Chia-Drop

(71) Applicant: The Jackson Laboratory, Bar Harbor, ME (US)

(72) Inventor: Yijun Ruan, Farmington, CT (US)

(21) Appl. No.: 18/007,129

(22) PCT Filed: Jul. 20, 2021
(86) PCT No.: PCT/US2021/042319

§ 371 Date: Jan. 27, 2023

Related U.S. Application Data
(60) Provisional application No. 63/058,088, filed on Jul. 29, 2020.

(51) Int. Cl. C12N 15/10; C12Q 1/6869

(52) U.S. Cl. CPC C12N 15/1065; C12Q 1/6869; C12Q 1/6806

(57) ABSTRACT

The scChIA-Drop method is a microfluidics-based dual-indexing strategy for single-cell and single-molecule chromatin interaction analysis.

### RELATED APPLICATIONS

This application claims the benefit under 35 U.S.C. § 119(e) of U.S. Provisional application Ser. No. 63/058,088, filed Jul. 29, 2020, the disclosure of which is incorporated by reference herein in its entirety.

### GOVERNMENT SUPPORT

This invention was made with government support under U54, DK107967 and UM1, HG009409 awarded by the National Institutes of Health. The government has certain rights in the invention.

### FIELD OF THE INVENTION

The invention, in some aspects, relates to methods of single-cell and single-molecule chromatin interaction analysis using droplet-based sequencing (ChIA-Drop).

### BACKGROUND

Droplet-based microfluidic approaches have been developed for single cell genomic (CNV), epigenomic (ATAC), and transcriptomic (RNA) analysis, but have not been developed for single cell chromatin interaction analysis. Multiplex chromatin interactions have previously only been inferred from ChIA-PET and Hi-C data based on daisy-chains of pairwise connectivity. Although an in-gel (polyacrylamide gel) method was attempted to explore multiple fragments in a chromatin complex by in-gel PCR [Gavrilov A. A., et al., Nucleic acids research. 2014, 42 (5): e36-10], there is no robust method to directly probe true complex chromatin interactions involving multiple loci simultaneously genome-wide. Single cell Hi-C [Nagano T, et al., Nature. 2013; 502(7469):59-64] still relies on conventional proximity ligation and standard molecular techniques-related work, and encounters the data sparsity issue inherent to all current single-cell genomic assays. Most existing single-cell data is only suitable for high-level profiling, and does not provide detailed molecular events of multiplex chromatin interactions at single-cell level. Studying complex chromatin interactions and directly mapping multiplex chromatin loops remains as a significant technical challenge.

### SUMMARY OF THE INVENTION

According to an aspect of the invention, a method of single-cell and single-molecule chromatin DNA barcoding is provided, the method including (a) preparing a plurality of encapsulated single cell beads; (b) generating single-cell barcoded chromatin DNA in the prepared encapsulated single cells; and (c) performing single-molecule chromatin DNA barcoding on the generated single-cell barcoded chromatin DNA, wherein the resulting barcoded chromatin DNA complex is single-cell/single-molecule barcoded chromatin DNA complex. In some embodiments, the method also includes (d) amplifying the single-cell/single-molecule barcoded chromatin DNA complex; (e) generating sequencing data from the amplified DNA sequences; and (f) analyzing one or more of the generated sequencing data arid the amplified DNA sequences. In certain embodiments, a means for preparing the plurality of encapsulated single-cell beads comprises mixing a microgel polymer and a single-cell suspension. In certain embodiments, a means of generating single-cell barcoded chromatin DNA complex in the prepared encapsulated single-cell beads, includes one or more of: (a) lysing the cells in the encapsulated single-cell beads; (b) digesting chromatin in cell nuclei in the encapsulated single-cell beads into chromatin fragments; and (c) generating single-cell droplets by combining the encapsulated single-cell beads that include the chromatin fragments with: (i) a plurality of a second gel bead including an independently selected single-cell-indexed DNA linker that includes a barcode indexed to the single cell; and (ii) a first reaction mix including a first independently selected enzyme; wherein: the generated single-cell droplets each includes one of the single-cell beads and one of the second gel beads; the second gel bead dissolves releasing the single-cell-indexed DNA linkers, and the released single-cell-indexed linkers are attached to the chromatin fragments forming single-cell-indexed barcoded chromatin DNA complexes. In some embodiments, a means of performing single-molecule chromatin DNA barcoding on the generated single-cell barcoded chromatin DNA, includes: one or more of: (a) pooling the cell nuclei and releasing the single-cell-indexed barcoded chromatin DNA complexes from the pooled nuclei; (b) generating a plurality of chromatin droplets by combining the released single-cell-indexed barcoded chromatin DNA complexes with: (i) a plurality of a third-gel bead that includes independently selected single-molecule-indexed DNA linkers that include a plurality of barcodes indexed for single-molecule barcoding and (ii) a second reaction mix that includes a second independently selected enzyme; wherein the generated chromatin droplets include the single-cell-indexed barcoded chromatin DNA complexes and one of the third-gel beads; the third-gel bead dissolves releasing the single-molecule-indexed DNA linkers; and the released single-molecule-indexed linkers are attached to an end of the chromatin fragments in the single-cell indexed barcoded chromatin DNA complexes forming chromatin DNA complexes that include a single-cell indexed barcode and a single-molecule-indexed barcode. In some embodiments, a means for digesting the chromatin includes a restriction enzyme digestion. In certain embodiments, the restriction enzyme digestion creates sticky DNA ends. In some embodiments, the restriction enzyme is a 4-bp cutter or a 6-bp cutter, wherein optionally the 4-bp cutter is MboI and optionally the 6-bp cutter is HindIII. In some embodiments, digesting the chromatin results in DNA fragments of 300-6000 bp. In certain embodiments, a means for digesting the chromatin includes a transposase digestion. In some embodiments, the transposase includes a Tn5 transposase polypeptide. In some embodiments, the transposase polypeptide is carrying an adapter DNA oligonucleotide for barcoding. In some embodiments, prior to combining the chromatin fragments with the plurality of single-cell-indexed barcoded linkers, the population of chromatin fragments is adjusted in solution to a solution concentration of 0.5 ng DNA/μl. In certain embodiments, prior to combining the chromatin fragments with the plurality of single-cell-indexed barcoded linkers the population of chromatin DNA complexes is enriched for a chromatin protein. In some embodiments, the enrichment includes incubating the population of chromatin fragments with a monoclonal antibody specific for the chromatin protein to form chromatin DNA complexes bound to the monoclonal antibody, isolating the chromatin DNA complexes bound to the monoclonal antibody, and removing the monoclonal antibody to form a population of chromatin DNA complexes each complex including the chromatin protein. In some embodiments, the chromatin protein is RNAPII, RARA, ER, or CTCF. in certain embodiments, the gel beads include gel beads in emulsion (GEMs), In certain embodiments, each GEM contains multiple copies of a DNA construct including a PCR priming site, a sequence reading site, one or both of a single-cell indexed barcode and a single molecule-indexed barcode, and a random priming nucleotide sequence. In some embodiments, the random priming nucleotide sequence is a random 8-mer. In some embodiments, one or both of the single-cell-indexed barcode and the single-molecule-indexed barcode include(s) ten or more nucleotides. In some embodiments, one or both of the single-cell-indexed barcode and the single-molecule-indexed barcode include(s) 8, 9, 10, 11, 12, or more nucleotides. In certain embodiments, one or both of the single-cell-indexed barcode and the single-molecule-indexed barcode include(s) a 15 nt to 25 nt barcode or a 16 nt to 20 nt barcode. In certain embodiments, the chromatin DNA complexes include chromatin DNA and chromatin protein. In some embodiments, a means of releasing the barcoded chromatin DNA complexes includes lysing the pooled nuclei. In some embodiments, the chromatin is released from the cell nuclei before digesting the chromatin into chromatin DNA fragments. In certain embodiments, a means for releasing the chromatin from the cell nuclei includes one or more of: crosslinking the nucleus with a crosslinking reagent, permeabilizing the crosslinked nucleus with a permeabilizing reagent, and digesting the permeabilized nucleus. In some embodiments, a means for lysing the single cell in the encapsulated single-cell bead includes: (a) crosslinking the single cell with a crosslinking reagent to form a crosslinked single cell that includes a crosslinked nucleus, (b) lysing the crosslinked single cell, (c) isolating the crosslinked cell nucleus from the lysed single cell, and (d) permeabilizing the isolated crosslinked cell nucleus with a permeabilizing reagent. In certain embodiments, the crosslinking reagent includes formaldehyde. In some embodiments, the formaldehyde is 1% (w/v) formaldehyde. In some embodiments, the permeabilizing reagent includes Sodium Dodecyl Sulphate (SDS). In some embodiments, the SDS is 0.5% SDS. In certain embodiments, the cross-linked permeabilized cell nucleus is fragmented by sonication prior to digestion. In some embodiments, a means of the amplifying the barcoded chromatin DNA includes isothermal incubation of the indexed single-cell and single-molecule barcoded chromatin DNA at about 30° C. for about 8-16 hours. In certain embodiments, one or both of the amplified indexed single-cell and single-molecule barcoded chromatin DNA fragments are subjected to one or more of end repair, A-tailing, and adapter ligation prior to sequencing. In certain embodiments, the sequencing is 150-bp sequencing. In some embodiments, the digesting step is performed using a restriction enzyme digestion. In some embodiments, the method also includes determining a chromatin. DNA interaction in the single cell at a single-molecule level.

According to another aspect of the invention, a method of single-cell and single-molecule chromatin DNA barcoding is provided, the method including: mixing a microgel polymer and a single cell/nuclei suspension to create a plurality of encapsulated single-cell beads; lysing the cells in the encapsulated single cell beads; digesting chromatin in the cell nuclei in the encapsulated single-cell beads into chromatin fragments; generating single-cell droplets by combining the encapsulated single-cell beads that include the chromatin fragments with: a plurality of a second gel bead that includes an independently selected single-cell-indexed DNA linker including a barcode indexed to the single cell; and a first reaction mix that includes a first independently selected enzyme; wherein: the generated single-cell droplets each include one of the single-cell beads and one of the second gel beads; the second gel bead dissolves releasing the single-cell-indexed DNA linkers, and the released single-cell-indexed linkers are attached to the chromatin fragments forming single-cell-indexed barcoded chromatin DNA complexes; pooling the cell nuclei and releasing the single-cell-indexed barcoded chromatin DNA complexes from the pooled nuclei; generating a plurality of chromatin droplets by combining the released single-cell-indexed barcoded chromatin DNA complexes with: a plurality of a third-gel bead that includes independently selected single-molecule-indexed DNA linkers that include a plurality of barcodes indexed for single-molecule barcoding and a second reaction mix that includes a second independently selected enzyme; wherein the generated chromatin droplets include the single-cell-indexed barcoded chromatin DNA complexes and one of the third-gel beads; the third-gel bead dissolves releasing the single-molecule-indexed DNA linkers; and the released single-molecule-indexed linkers are attached to an end of the chromatin fragments in the single-cell indexed barcoded chromatin DNA complexes forming chromatin DNA complexes that include a single-cell indexed barcode and a single-molecule-indexed barcode; (g) amplifying the barcoded chromatin DNA; (h) generating sequencing data from the amplified DNA sequences; and (i) analyzing one or more of the generated sequencing data and the amplified DNA sequences. in certain embodiments, a means for digesting the chromatin in step (c) includes a restriction enzyme digestion. In some embodiments, the restriction enzyme digestion creates sticky DNA ends. In some embodiments, the restriction enzyme is a 4-bp cutter or a 6-bp cutter, wherein optionally the 4-bp cutter is MboI and optionally the 6-bp cutter is HindIII. In some embodiments, digesting the chromatin results in DNA fragments of 300-6000 bp. In certain embodiments, a means for digesting the chromatin in step (c) includes a transposase digestion. In certain embodiments, the transposase includes a Tn5 transposase polypeptide. in some embodiments, the transposase polypeptide is carrying an adapter DNA oligonucleotide for barcoding. In some embodiments, prior to combining the chromatin fragments with the plurality of single-cell-indexed barcoded linkers, the population of chromatin fragments is adjusted in solution to a solution concentration of 0.5 ng DNA/μl. In certain embodiments, prior to combining the chromatin fragments with the plurality of single-cell-indexed barcoded linkers the population of chromatin DNA complexes is enriched for a chromatin protein. In some embodiments, the enrichment includes incubating the population of chromatin fragments with a monoclonal antibody specific for the chromatin protein to form chromatin DNA complexes bound to the monoclonal antibody, isolating the chromatin DNA complexes bound to the monoclonal antibody, and removing the monoclonal antibody to form a population of chromatin DNA complexes each complex including the chromatin protein. In some embodiments, the chromatin protein is RNAPII, RARA, ER, or CTCF. In certain embodiments, the gel beads include gel beads in emulsion (GEMs). In some embodiments, each GEM contains multiple copies of a DNA construct that includes a PCR priming site, a sequence reading site, one or both of a single-cell indexed barcode and a single molecule-indexed barcode, and a random priming nucleotide sequence. In some embodiments, the random priming nucleotide sequence is a random 8-mer. In certain embodiments, one or both of the single-cell-indexed barcode and the single-molecule-indexed barcode include(s) ten or more nucleotides. In certain embodiments, one or both of the single-cell-indexed barcode and the single-molecule-indexed barcode include(s) 8, 9, 10, 11, 12, or more nucleotides. In some embodiments, one or both of the single-cell-indexed barcode and the single-molecule-indexed barcode include(s) a 15 nt to 25 at barcode or a 16 nt to 20 nt barcode. In some embodiments, the chromatin DNA complexes include chromatin DNA and chromatin protein. In some embodiments, a means of releasing the barcoded chromatin DNA complexes includes lysing the pooled nuclei. In certain embodiments, the chromatin is released from the cell nuclei before digesting the chromatin into chromatin DNA fragments. In some embodiments, a means for releasing the chromatin from the cell nuclei includes one or more of: crosslinking the nucleus with a crosslinking reagent, permeabilizing the crosslinked nucleus with a permeabilizing reagent, and digesting the permeabilized nucleus. In some embodiments, a means for lysing the single cell in the encapsulated single-cell bead includes: (a) crosslinking the single cell with a crosslinking reagent to form a crosslinked single cell that includes a crosslinked nucleus, (b) lysing the crosslinked single cell, (c) isolating the crosslinked cell nucleus from the lysed single cell, and (d) permeabilizing the isolated crosslinked cell nucleus with a permeabilizing reagent. In certain embodiments, the crosslinking reagent includes formaldehyde. In some embodiments, the formaldehyde is 1% (w/v) formaldehyde. In some embodiments, the permeabilizing reagent includes Sodium Dodecyl Sulphate (SDS). In certain embodiments, the SDS is 0.5% SDS. In certain embodiments, the cross-linked permeabilized cell nucleus is fragmented by sonication prior to digestion. In some embodiments, a means of the amplifying the barcoded chromatin DNA includes isothermal incubation of the indexed single-cell and single-molecule barcoded chromatin DNA at about 30° C. for about 8-16 hours. In some embodiments, one or both of the amplified indexed single-cell and single-molecule barcoded chromatin DNA fragments are subjected to one or more of end repair, A-tailing, and adapter ligation prior to sequencing. In some embodiments, the sequencing is 150-bp sequencing. In certain embodiments, the digesting step is performed using a restriction enzyme digestion. In certain embodiments, the method also includes determining a chromatin DNA interaction in the single cell at a single-molecule level.

According to another aspect of the invention, a method of mapping chromatin DNA complexes is provided, the method including (a) determining the amplified DNA sequences as set forth in any embodiment of any of the aforementioned aspects of the invention, and (b) analyzing the amplified DNA sequences. In some embodiments, a means of analyzing the amplified DNA sequences includes a ChIA-DropBox pipeline method.

According to another aspect of the invention, a method of ChIA-DropBox pipeline sequence analysis is provided, the method including: (a) reading the sequence data generated using a method as set forth in any embodiment of any of the aforementioned aspects of the invention; (b) identifying one or more of the barcodes on the barcoded chromatin DNA based on the reading; (c) calling of GEMS based on the barcode identification; (d) identifying significant chromatin DNA complexes; and (e) visualizing the data obtained in (d).

According to another aspect of the invention a method of a single-cell chromatin identification is provided the method including: (a) preparing a plurality of single-cell gel beads, each including a cell nucleus of a single cell, wherein the cell nucleus includes chromatin DNA complexes; (b) digesting the chromatin DNA complexes into chromatin DNA fragments; (c) mixing the single-cell gel beads that include the chromatin DNA fragments with: (i) a plurality of a second gel bead, each including a plurality of an indexed barcode linker including a barcode indexed to the single cell; and (ii) reagents that include an enzyme capable of ligating the barcodes to the chromatin DNA fragments, (d) partitioning the single-cell gel beads and the second gel beads in the mixture into individual single-cell droplets that include at least one of the single-cell gel beads and at least one of the second gel beads; and (e) releasing the indexed barcode linkers within each single-cell droplet, wherein the released single-cell indexed barcode linkers add one of the indexed single-cell barcodes to a chromatin DNA fragment in the single-cell droplet, thereby generating indexed single-cell barcoded chromatin DNA fragments, wherein the chromatin DNA from the single cell is identified by the presence of the chromatin DNA fragments including the indexed single-cell barcode. In some embodiments, the method also includes determining a chromatin DNA interaction in the single cell at a single-molecule level. In certain embodiments, a means of determining the chromatin DNA interaction at the single molecule level includes a Chia-PET, Hi-C, or a ChIA-drop method.

### BRIEF DESCRIPTION OF THE DRAWINGS

FIG. 1 provides a schematic diagram of an embodiment of a ChIA-Drop method.

FIG. 2A-C shows schematic diagrams of single-cell and single-molecule chromatin barcoding strategy using the Chromium microfluidics system. FIG. 2A illustrates how microgel polymer and single-cell (single-nucleus) suspension are mixed to create encapsulated single-cell beads, followed by cell lysis and chromatin digestion by restriction enzyme or transposase within each cell bead. FIG. 2B illustrates how cell beads are then combined with gel beads that each deliver a DNA linker with a unique barcode and a reaction mix containing enzyme to generate single-cell droplets containing one cell bead and one gel bead. Once partitioned, the gel bead dissolves and releases DNA linker barcodes within each droplet. The DNA linker is added to the end of each chromatin fragment, generating single-cell-specific barcoding. FIG. 2C illustrates how barcoded nuclei are pooled and lysed. The released chromatin DNA complexes bearing nuclear-specific barcodes are partitioned with another set of gel-beads and reaction mix including enzyme for single-molecule barcoding in chromatin droplets. The chromatin droplets are isothermally incubated to amplify the barcoded chromatin DNA fragments for ChIA-Drop sequence analysis. The resulting scChIA-Drop reads are expected to contain both of the nuclear-specific barcodes and the molecule-specific barcodes for downstream processes.

### DETAILED DESCRIPTION

Aspects of the invention provide single-cell CAA-Drop (scChIA-Drop) methods that achieve robust single-molecule detection of multiplex chromatin interactions in single nuclei and in bulk cells. Certain embodiments of scChIA-Drop methods of the invention comprise separating individual cell nuclei and individual molecules of chromatin DNA complexes in a massively parallel manner in large numbers of droplet-based reactions for detection of multiplex chromatin interactions with single-cell specificity and single-molecule resolution. This approach enables direct detection of multi-way interactions (protein-RNA-chromatin-chromatin), which has not been achieved with previous methods, and provides opportunities for studying a wide range of biomedical questions. Single-cell ChIA-Drop methods of the invention are cost-effective and require fewer input cells than conventional approaches due to the practicality, simplicity, and robustness of scChIA-Drop methods. Single-cell ChIA-Drop methods of the invention can be used to examine the multiplexity of chromatin interaction biology and their use permits significant advances in understanding of chromatin topological structures and specific genome regulatory functions, including transcription regulation.

Certain embodiments of methods of the invention include use of a droplet-based and barcode-linked microfluidics system for single cell and single molecule detection of complex chromatin interactions. A platform and methods have now been developed for single-cell and single-molecule capability of ChIA-Drop that can be used to identify multivalent and combinatorial chromatin interactions simultaneously associating with chromatin architecture proteins and regulatory RNAs. Certain embodiments of methods of the invention can be used to identify such interactions and the identification can be used in methods to determine chromatin topology and genome functions in healthy and diseased cells.

As described herein, in some embodiments an scChIA-Drop method comprises single-cell/nucleus barcoding and single-molecule barcoding. This dual-indexing strategy (nucleus-specific and chromatin-specific) is used to achieve simultaneous detection of single-cell and single-molecule chromatin interaction analysis without physical isolation of single nuclei and single chromatin molecules. Individual nuclei are barcoded (nuclear indexing) and the nuclear-indexed chromatin samples are partitioned for droplet-specific barcoding in ChIA-Drop analysis.

Combining single-molecule and single-cell ChIA-Drop in methods of the invention results in robust scChIA-Drop data that can be used to determine multiplex genomic loci that are simultaneously interacting with each other in individual cells, which has not been possible using prior 3D genome mapping technologies. Certain embodiments of a single-cell ChIA-Drop method of the invention comprises both scChIA-Drop and single-molecule ChIA-Drop methods. Some embodiments of the invention include scChIA-Drop methods and do not include single-molecule ChIA-Drop methods. In each case, single-cell ChIA-Drop methods of the invention can be used to advance the field of 3-D genome biology and to understand and answer biomedical questions.

### ChIA-Drop

ChIA-Drop methods of the invention provide true and robust detection of multiplex chromatin interactions in single nuclei and in single cells. Embodiments of the invention comprise separating individual cell nuclei and individual molecules of chromatin DNA complexes in a massively parallel manner in a plurality of droplet-based reactions (for example, though not intended to be limiting, picoliter reactions) for detection of multiplex chromatin interactions with single-cell specificity and single-molecule resolution. The terms “single cell” and “single nuclei” may be used interchangeably in descriptions of aspects of the invention. As non-limiting examples, (1) in methods comprising a “single-cell suspension”, the “single-cell suspension” would be understood to be equivalent to a “single-nuclei suspension” because the single nuclei are the nuclei of the single cells; (2) a plurality of encapsulated single-cell beads may also be referred to as a plurality of encapsulated single-cell/single-nuclei beads, or as a plurality of encapsulated single-nuclei beads; and (3) a single-cell/single molecule barcoded chromatin DNA complex may be referred to as a “single-nuclei/single molecule barcoded chromatin DNA complex.

In some embodiments ChIA-Drop methods of the invention can be applied samples that include least 1, 10, 100, 1,000, 10,000, 100,000, 500,000, 1,000,000, 5,000,000, or more cells. Methods of the invention comprise technical elements such as: (i) microfluidics to multiplex chromatin interactions (which is distinct from prior microfluidic applications for single-cell RNA and DNA sequencing); (ii) unique dual barcoding (nuclear-specific and complex-specific chromatin barcoding) strategies to achieve simultaneous detection of single-nucleus and single-molecule chromatin DNA complexes without requiring their physical separation; and (iii) eliminating proximity ligation steps and including direct use (not purified) of chromatin DNA fragments in droplets for ChIA-Drop analysis.

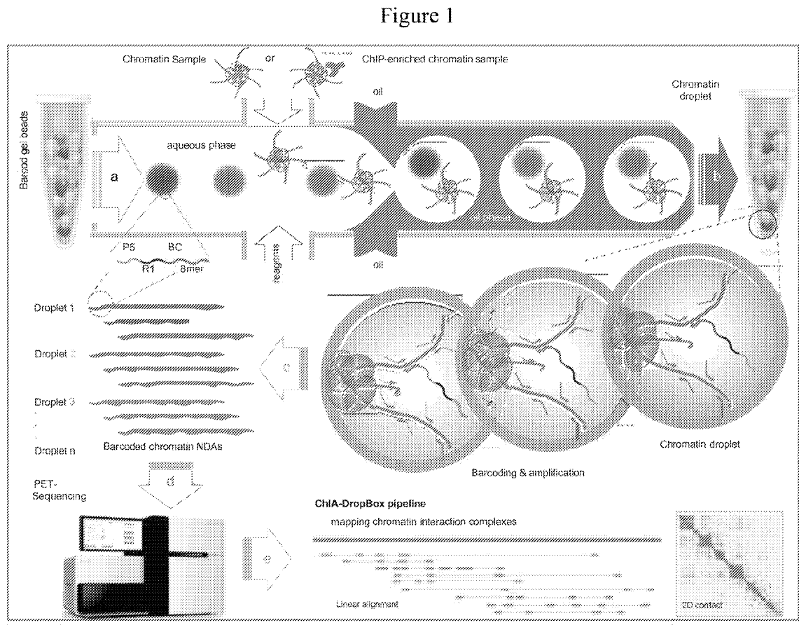

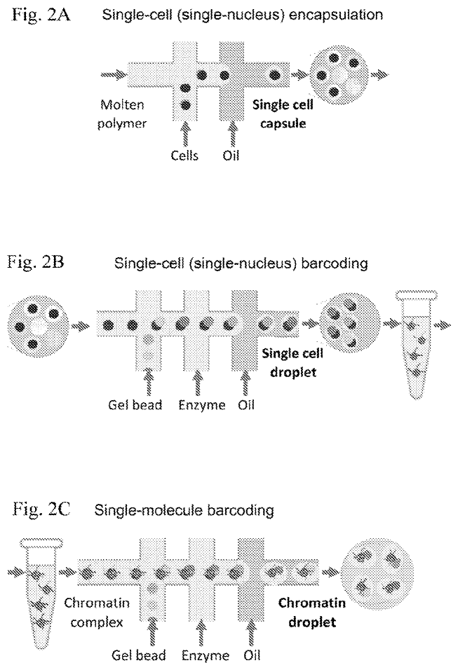

Certain embodiments of methods of the invention can be used to achieve molecule-specific indexing for analysis of multiplex chromatin interactions with single molecule precision (FIG. 1) [Zheng M, et al, Nature. 2019 February; 566(7745):558]. ChIA-Drop methods of the invention include, in part, use of hydrogel-beads for two levels of DNA indexing to barcode chromatin fragments in a nucleus-specific and molecule-specific manner (FIG. 2A-C). Certain embodiments of scChIA-Drop methods of the invention comprise elements of single-molecule ChIA-Drop methods [see Zheng, M. et al., (2019) Nature. February; 566(7745): 558-562]. Certain embodiments of scChIA-Drop methods of the invention include three main aspects: (1) single-cell/single-nucleus encapsulation, (2) single-cell chromatin barcoding, and (3) single-molecule chromatin barcoding (see FIG. 2A-C). First, single cells (nuclei) are individually encapsulated by microgel polymer, followed by in situ chromatin digestion in each single cell capsule, either by restriction enzyme to create sticky DNA ends for later DNA linker ligation, or by Tn5 transposase carrying adapter DNA oligos for later DNA barcoding. The single cell capsules are combined with hydrogel beads (each bead comprising many copies of DNA oligo linker with bead-specific barcode) and a reaction mix containing enzyme to form droplets of single-cell with a gel bead-in emulsions (GEM). In some embodiments, a droplet comprises one single-cell capsule and one hydrogel-bead per droplet. Once partitioned, the hydrogel-bead dissolves, releasing DNA linker barcodes and enzymatically indexing chromatin fragments in each single cell droplet. The droplets are then “broken” and release the nuclear-barcoded chromatin material. The mix of nuclear-barcoded chromatin DNA complexes are partitioned through microfluidics with another set of hydrogel-beads and reaction mix including enzyme to form droplets with single molecule of chromatin DNA complex with gel bead-in emulsions for single molecule chromatin barcoding, wherein in some embodiments of the invention the droplet comprises one chromatin DNA complex and one hydrogel-bead. The chromatin droplets are isothermally incubated to amplify the barcoded chromatin DNA fragments as illustrated in FIG. 1, resulting in what is referred to herein as an scChIA-Drop library. In some embodiments of the invention, all or part of the resulting scChIA-Drop library may be sequenced and analyzed [Zheng M, et al, Nature. 2019 February; 566(7745):558].

Single-cell ChIA-Drop reads resulting from embodiments comprising dual-indexing methods, contain both the nucleus-specific barcode and the molecule-specific barcode, thereby achieving scChIA-Drop analysis with single-molecule precision. Embodiments of methods of the invention provide a means in which a hydrogel-bead barcoding system is used twice, first for the cell/nucleus-specific indexing, and second for the chromatin-specific indexing. In some embodiments of the invention, a hydrogel bead comprises a DNA oligo with a total barcode capacity of four million (4×10⁶) indexes. In some embodiments of the invention, a set of the hydrogel-beads prepared using methods of the invention may comprise millions (10⁶) of bead-specific unique barcodes, and the random combinations of the nuclear-specific and molecule-specific indexing may generate trillions (10¹²) of indexing capacity. Single-cell ChIA-Drop methods of the invention, including, but not limited to methods comprising indexed single-molecule barcoding and indexed single-cell barcoding are cost-effective and require fewer input cells than prior approaches clue to their high efficiency and robust results.

### CNA-Drop General Procedures

Single-cell ChIA Drop methods of the invention can be utilized to achieve low-cost, rapid, and high-quality data generation. In contrast to prior chromatin sample preparation, which was originally established for proximity ligation, Single-cell ChIA-Drop methods of the invention do not require a ligation step, thus simplifying chromatin preparation in the cell lysis and chromatin fragmentation steps. Some embodiments of scChIA-Drop methods of the invention include crosslinking conditions without ChIP enrichment, and certain embodiments of scChIA-Drop methods include double crosslinking conditions with ChIP-enrichment. Certain embodiments of the invention comprise enzymatic digestions, including but not limited to restriction enzyme digestion at specific sites, MNase for random cleave, and transposase.

Testing ChIA-Drop libraries made from chromatin samples, may in some embodiments of the invention be analyzed using means such as, but not limited to small-scale MiSeq sequencing. Such methods can be used to assess quality of scChIA-Drop libraries prepared using methods of the invention. In some embodiments of the invention a set of quality assurance/quality control (QA/QC) measurements of sequencing data generated using methods of the invention, such as but not limited to: fragment read length, size distribution of chromatin DNA complex (number of fragments per complex) may be analyzed. In some embodiments, methods of the invention include loading chromatin samples for preparing droplets, a non-limiting example of which is loading chromatin to Chromium Controller (10X Genomics) for droplet making. In some embodiments of the invention, the number of chromatin particles partitioned into microfluidic droplets follows the Poisson distribution, and the quantity of the loaded chromatin sample is optimized to reach the Poisson rate 1, which for example, maximizes the number of droplets with a single chromatin DNA complex, and minimizes the number of empty droplets and/or mixed droplets.

Microfluidic protocols are utilized that optimize droplet formation and include molecular reagents, the selection of which is tailored at least in part, for chromatin interaction analysis. In some embodiments of the invention, methods include preparing droplets having a balance between the number of the droplets and the size of the droplets. For example, though not intended to be limiting, in some embodiments of the invention, a scChIA-Drop workflow comprises use of a higher numbers of droplets of smaller size as compared to a workflow comprising fewer droplets of larger size. Methods of the invention comprise droplets of preselected number and size used in conjunction with high amplification rates for chromatin DNA fragments.

The number of cells encapsulated in a scChIA-Drop method of the invention may be selected, at least in part, based on amount of chromatin DNA material to be used to prepare an scChIA-Drop library from the cells. In some embodiments of the invention, the amount of chromatin DNA used to prepare an scChIA-Drop library may be as low as 0.01 ng, 0.05 ng, 0.1 ng, 0.2 ng, 0.3 ng, 0.4 ng 0.5 ng, 0.6 ng, 0.7 ng, 0.8 ng, 0.9 ng, or 1.0 ng. An advantage of the small amount of chromatin DNA needed in scChIA-Drop methods of the invention, is that scChIA-Drop methods can use relatively low numbers of cells as starting material to prepare the scChIA-Drop library. In certain embodiments of scChIA-Drop methods of the invention the number of cells encapsulated may be up to: 100, 500, 1,000, 5,000, 10,000, 20,000, 30,000, 40,000, 50,000, 60,000, 70,000, 80,000, 90,000, 100,000, 500,000, 1,000,000, 2,000,000, or 5,000,000 cells, including all integers therein. In some embodiments of the invention, a plurality of cells may include a number of cells in a range from 500-5,000 cells, 500-10,000 cells, 500-50,000 cells, 500-100,000 cells, 500-500,000, 1,000-10,000, 5,000-50,000, 10,000-100,000, 50,000-500,000, 100,000-1,000,000, or 500,000-5,000,000 including all integers in the ranges, in some embodiments of methods of the invention larger numbers of cells are encapsulated and used to prepare an scChIA-Drop library, for example, in some instances at least 1,000,000; 2,000,000; 3,000,000; 4,000,000; 5,000,000; or more cells can be in a plurality of cells used in a method of the invention. Thus, certain embodiments of scChIA-Drop methods may include preparing a plurality of encapsulated cells/nuclei wherein the number of cells is in excess of a number needed to for successful ChIA-Drop library construction.

### Determining Chromatin Interactions

In certain aspects of the invention methods of determining a chromatin interaction at a single cell level are provided, and in some aspects of the invention methods of determining a chromatin interaction at a single cell single and single molecule level are provided. As used herein the term “determining” used in relation to a chromatin interaction means identifying chromatin interactions at the single-molecule level. The ability to identify such interactions at the level of single molecules provides an advantage over prior pairwise, composite methods such as Hi-C and ChIA-PET methods. Methods of the invention have been used to confirm the presence of simultaneous multiplex chromatin interactions on the same chromatin string, and that the chromatin DNA complexes within the same topological domains are highly heterogeneous, indicating a high level of variation in chromatin contacts at the single molecule level in cells.

Certain embodiments of methods of the invention includes preparing single-cell beads, which may also be referred to herein as single-cell capsules. The terms “single-cell” and “single-nuclei” and “single cell/nucleus” may be used interchangeably herein with respect to such beads and capsules. Single-cell beads for use in methods of the invention may be prepared. by mixing a microgel polymer and single cell/nucleus suspension to create encapsulated single-cell beads. A non-limiting example of a microgel polymer that can be used to prepare encapsulated single-cell beads is molten agarose. Additional art-known microgel polymers may be used to prepare single-cell beads.

In certain embodiments of the invention, encapsulated single cells/nuclei are permeabilized and incubated with reaction mix for in situ chromatin digestion. Non-limiting examples of reaction mixes for in situ chromatin digestion are a reaction mix comprising HindIII to create sticky DNA ends for later DNA linker ligation and a reaction mix comprising Tn5 transposase carrying adapter DNA oligos for later DNA barcoding.

The digested chromatin fragments in each nucleus in a prepared single-cell bead may be processed in a manner that results in single-cell (single-nucleus) barcoding. In some embodiments of the invention, a fragmented chromatin sample is directly applied to a microfluidics system, and each chromatin DNA complex is compartmentalized in a Gel-bead-in-Emulsion (GEM) droplet that contains unique DNA oligonucleotides and reagents for linear amplification and barcoding of chromatin DNA templates. The barcoded amplicons with GEM-specific indices may be pooled for standard high-throughput sequencing, and the sequencing reads with identical barcodes are assigned to the same GEM of origin, indicating they are derived from the same chromatin DNA complex. Mapping of the DNA sequencing reads to a reference genome identifies which remote genomic loci were in close spatial proximity. Based on these mapped loci, multiplex chromatin interactions can be detected.

In some embodiments of the invention, prepared single-cell beads are combined with hydrogel beads and a reaction mix containing enzyme and gel-bead-in-emulsion (GEM) droplets is formed that comprise one single-cell capsule and one hydrogel bead per droplet. Thus, in some embodiments of the invention single-cell droplets are generated by combining the encapsulated single-cell beads comprising the chromatin fragments with a plurality of a second gel bead that comprises an independently selected single-cell-indexed DNA linker that comprises a barcode indexed to the single cell; and a first reaction mix that includes a first independently selected enzyme. In some embodiments of the invention, single-cell droplets are generated using a microfluidic device, a non-limiting example of which is a Nadia Innovate microfluidics device (Dolomite Bio, Royston, UK). Single-cell droplets generated in this manner each comprise one of the single-cell beads and one of the second gel beads. After the single-cell droplets are generated, the second gel bead dissolves releasing the single-cell-indexed DNA linkers, and the released single-cell-indexed linkers attach to the chromatin fragments thereby forming single-cell-indexed barcoded chromatin DNA complexes.

### Chromatin Preparation

In some embodiments of the invention, a sample used in an scChIA-Drop method is obtained from a crosslinked, permeabilized cell nucleus, which is digested to provide a population of chromatin DNA complexes. A chromatin DNA complex is comprised of chromatin DNA and chromatin protein. Methods of crosslinking a cell nucleus are known in the art, and in certain embodiments of the invention, an scChIA-Drop method includes use of live cells, such as tissue culture cells or cell isolated from freshly dissected tissues. In certain embodiments, the cell nucleus of the live cell is cross-linked using a fixative such as one or more of formaldehyde- and EGS (Ethylene glycol bis[succinimidylsuccinate]). Other art-known crosslinking reagents suitable for crosslinking protein-DNA, protein-RNA and/or protein-protein (e.g., those having two or more reactive chemical groups suitable for reacting with the amide and/or thiol groups) may also be used. If EGS is used, a spacer region between the two NHS-esters may be a 12-atom spacer, although longer or shorter spacers (e.g., 6, 7, 8, 9. 10, 11, 12, 13, 14, 15, 16, 17, 18, 19, 20 atom spacers) may be used as well. If formaldehyde or EGS (typically about 1-2 mM, or 1.5 mM) are used, EGS may be added first followed by (about 1%) formaldehyde. Reaction may be quenched by glycine. Alternatively, about 1% formaldehyde or about 1% glutaraldehyde may be used. In a non-limiting example, about 1-2×10⁸ live tissue culture cells or isolated cells are collected and cross-linked with EGS with shaking for 40 min., then contacted with formaldehyde (final concentration of about 1%) for 10 minutes at room temperature. In some aspects of the invention, the formaldehyde is greater than 0.5% (w/v) formaldehyde, In certain embodiments the formaldehyde is about 1% (w/v) formaldehyde.

An alternative cross-linking means that may be used in certain embodiments of the invention comprises UV cross-linking. In a non-limiting example, tissue culture cells may be UV-crosslinked at about 150 mJ/cm² at 254 nm, a non-limiting example of which is a UV crosslinker, such as STRATALINKER® UV crosslinker. Additional art-known means of cross-linking may also be suitable for use in an embodiment of the invention. Cross-linking methods are described, see for example: Li, X, et al., Nat. Protoc. 2017 May: 12(55):899-915; US Patent Pub. 2016/0177380; and Belton, J, et al. Methods, 2012 November: 58(3), the content of each of which is incorporated herein by reference in its entirety by reference.

Following cross-linking of the nucleus, the cross-linked nucleus is permeabilized using a methods such as contact with SDS or other suitable agent. In some embodiments, a proteinase inhibitor and/or RNase inhibitor may be added to the sample to prevent non-specific proteinase or RNase digestion. Cell lysis is then carded out using a suitable lysis buffer, a non-limiting example of which includes SDS. For example, a lysis buffer may comprise: 50 mM HEPES, 1 EDTA, 0.15 M NaCl, 1% SDS, 1% Triton X-100, and 0.1% sodium deoxycholate. Other suitable lysis buffers may also be used and are known in the art. See for example: Li, X, et al., Nat. Probe. 2017 May: 12(55):899-915; US Patent Pub, 2016/0177380; and Belton, J. et al, Methods, 2012 November: 58(3), the content of each of which is incorporated herein by reference in its entirety by reference.

In some embodiments of the invention, chromatin fragments are generated by physical shearing, such as sonication, hydroshearing, or repeated drawing through a hypodermic syringe needle. Sonication means may be used to break up chromatin fibers. In some embodiments of the invention chromatin fragments may be generated using restriction enzyme digestion, or partial or limited endo- and/or exo-nuclease digestion. Various different commercially available instruments are suitable for sonication. For example, the 5220 Focused-ultrasonicator from Covaris, Inc. utilizes the Adaptive Focused Acoustics™ (AFA) technology for DNA, RNA, and chromatin shearing, and the BIORUPTOR® UCD-200 (Life Technologies Corp.) may also be used. After shearing, the chromatin may be diluted (for example, at least 2×, 3×, 4×, 5×, 6×, 7×, 8×, 9× or, 10 times) to lower the SDS concentration (for example, to about 0.1-0.5%). The extract may be cleared by centrifugation (a non-limiting example of which is at 14,000 rpm for 10 minutes at 4° C.). The resulting extract can be stored at −80° C. until use.

Following the sonication process the resulting material may be digested using one or more restriction enzymes. In some aspects of the invention the restriction enzyme is a 4 bp cutting enzyme such as, but not limited to: MboI. In certain aspects of the invention the restriction enzyme is a 6-bp cutting enzyme such as, but not limited to: HindIII. Additional art-known restriction enzymes may also be used in embodiments of the invention, for example, other 4-bp or 6-bp cutters, or other length cutters such as a 5-bp or 7-bp cutter, etc. One skilled in the art will be able to identify and use suitable alternative restriction enzymes in methods of the invention. In certain aspects of the invention, the digestion provides DNA fragments of 300-6000 bp in the chromatin DNA complex.

### Chromatin Sample Preparation for ChIA-Drop

A non-limiting example of a method of preparing a chromatin sample for use in an scChIA-Drop method of the invention is as follows, a process that is similar to sample preparation for Hi-C2, but the scChIA-Drop method need not include proximity ligation. In this non-limiting example: ˜10 million cells were crosslinked for 10 min in 1% formaldehyde at room temperature. The crosslinked cells were quenched for 5 min in 0.125 M Glycine, and were then washed twice with L)PBS twice. The crosslinked cells can be stored at −80° C. for later use or processed immediately with procedures for cell/nuclei lysis. For cell/nuclei lysis, crosslinked cells were suspended in 500 μl of cell lysis buffer (10 mM Tris-HCl pH 7.0, 10 mM Tris-HCl pH 8.0, 10 mM NaCl, 0.2% NP40, 1× Protease Inhibitor cocktail, Roche) and incubated at 4° C. for 30 min with rotation. The nuclei are isolated by centrifugation at 4° C. for 5 min at 2,500 relative centrifugal force. The nuclei pellet can be suspended in 100 μl of 0.5% SDS and incubated for 5 minutes at 62° C. to permeabilize the nuclear membrane. Following permeabilization, 285 μl of nuclease-free water and 25 μl of 20% triton X-100 can be added for and the mixture incubated for 15 min at 37° C. to neutralize SDS.

The permeabilized nuclei were then processed using in situ chromatin digestion. When digested by a 4-bp cutter MboI, 60 μl of NEB Buffer 2 was added to the permeabilized nuclei and mixed well. 55 μl of nuclease-free water and 75 μl of MboI (5 U/μl) can be added to the mixture. In embodiments in which a 6-bp cutter digestion HindIII is performed, 80 μl of nuclease-free water and 50 μl of HindIII (20 U/μl) can be added to set up the reactions. The reactions that included either the 4 bp cutter or 6 bp cutter are incubated overnight at 37° C. with constant agitation. The nuclei with digested chromatin materials are then sheared by sonication with 1× Protease inhibitor cocktail to release the chromatin fragments. The DNA size range of the chromatin fragments generally was in the range of about 300-6000 bp, depending on restriction digestion. The final fragmented chromatin sample is utilized for scChIA-Drop library construction.

### Enriched Chromatin Population

In certain aspects of the invention, a population of chromatin DNA complexes is an enriched population. In some aspects of the invention the chromatin DNA complex population is enriched for a chromatin protein by incubating the population of chromatin DNA complexes with a monoclonal antibody specific for the chromatin protein in order to form chromatin DNA complexes bound to the monoclonal antibody. Different chromatin proteins may be of interest for enrichment, for example, though not intended to be limiting, the chromatin protein that is enriched is RNAPII, Retinoic acid receptor alpha (RARA), ER, or transcriptional repressor protein CTCF, also known as 11-zinc finger protein or CCCTC-binding factor. Other chromatin proteins may be of interest for enrichment and methods and monoclonal antibodies, or functional fragments thereof that are suitable for use in enrichment can he used in embodiments of the invention fur chromatin protein enrichment. Following the binding of a monoclonal antibody of interest to the chromatin DNA complexes, the bound chromatin DNA complexes bound are isolated and the monoclonal antibody is removed, which results in a population of chromatin DNA complexes in which each complex comprises the chromatin protein. As a non-limiting example of an enrichment process: 2 μg of a monoclonal antibody of interest that is specific for a chromatin component is bound to a substrate (for example protein C sepharose). The antibody-coated beads are incubated with the chromatin extract and the beads are washed. The resulting protein-DNA complexes are eluted from the beads with elution buffer and the eluent is then dialyzed to remove SDS.

A non-limiting example of a method of preparing a chromatin sample for use in a RNAPII enriched scChIA-Drop method of the invention is as follows, a process that is similar to sample preparation for Hi-C2, except the method of the invention does not include proximity ligation. In the example, cells in a plurality of cells are dual-crosslinked with 1.5 mM EGS for 40 min followed by 1% formaldehyde reaction for 20 min, and then quenched with 0.125 M Glycine (Promega) for 10 min, and washed twice with DPBS. After cell and nuclei lysis, the crosslinked chromatin material is fragmented by sonication to the size range of 6 kb. The fragmented chromatin sample is incubated overnight with 20 μl of anti-RNAPII monoclonal antibody bound on Dynabeads™ Protein G beads at 4° C. with rotation. RNAPII-enriched chromatin is released from Protein G beads by incubating for 30 min with EB Buffer containing 1% SDS at 37° C. with constant agitation. The elution supernatant is passed through Ultra Centrifugal Filter to remove remaining SDS. The chromatin preparation now is ready for ChIA-Drop library construction, or to be stored at 4° C. for later use. It will be understood that the above solutions and procedure are included as example and that other art-known buffers, antibodies, and procedures are suitable for use in enrichment methods of the invention.

### Processing ChIA-Drop Data

Certain embodiments of the invention utilized the R statistical package (r-project.org/) for statistical analyses. Certain terminology used herein includes the term “gene promoter” which as used herein means a region that is ±250 bps of the Transcription Start Site (TSS) of a gene including all isoforms. As used herein a gene is indicated as “active” if its RNA-Seq expression level RPKM≥1 and “inactive” if it has an RPKM<1. As used herein a promoter of an active gene is referred to as “active promoter” and that of an inactive gene is referred to as “inactive promoter”. As included herein, all regions outside of gene promoter regions are “non-promoter” (or “enhancer”) regions. Certain terms used herein including: “Topologically Associating Domain” and “RNAPII Associated Interaction Domain” are abbreviated as “TAD” and “RAID”, respectively.

### ChIA-DropBox Data-Processing Pipeline

As used herein a data processing pipeline, referred to as ChIA-DropBox, has been developed and is comprehensive data-processing pipeline that can be used to convert. ChIA-Drop raw reads into meaningful chromatin interaction data. Thus, in some aspects of the invention methods such as the ChIP-DropBox procedure may be used to analyze and map the sequenced chromatin DNA from which a plurality of DNA sequence reads have been generated.

As a first step in ChIP-Dropbox, reads are aligned to the reference genome (dm3) using the 10X Genomics longranger wgs pipeline (v2.1.5, see: //support.10xgenomics.com/genome-exome/software/pipelines/latest/using/wgs), from which GEMcodes are identified with pysam module (v0.7.5) in python (v2.7.13). Uniquely mapped reads with MAPQ≥30 and read length≥50 by are extended by 500 bps from its 3′ end, and those with the same GEMcode overlapping within 3 kb distance are merged using pybedtools (v0.7.10). Multiplexed intra-chromosomal GEMs are retained as potential chromatin DNA complexes, and their statistical significances are estimated by comparing fragment distances to a null distribution of randomly rewired. GEMs (see Examples section for more details).

A process, such as the ChIA-DropBox process also permits visualization of ChIA-Drop data in various types/formats: 1) 2D heatmap via. Juicer tools (v1.7.5) and Juicebox (v1.9.0; v1.1.2); 2) pairwise loops; and 3) linear fragment alignments. Full details of ChIA-DropBox and ChIP-DropBox analysis that can be used in methods of the invention are available in the art. Additional art-known processing methods are suitable for use in embodiments of methods of the invention.

### Cells

It will be understood that a cell sample used in a method of the invention comprises a plurality of cells. As used herein the term “plurality” means more than one. In some instances a plurality of cells is least 1, 10, 100, 1,000, 10,000, 100,000, 500,000, 1,000,000, 5,000,000, or more cells. A plurality of cells included in a sample used in a method of the invention may be a population of cells. A plurality of cells may include cells that are of the same cell type. In some embodiments of the invention, a plurality of cells includes cells having a known or suspected disease or condition. In some embodiments of the invention, a plurality of cells is a mixed population of cells, meaning all cells are not of the same cell type. A cell used in a method of the invention, may be obtained from a biological sample obtained directly from a subject. Non-limiting examples of biological samples are samples of: blood, saliva, lymph, cerebrospinal fluid, vitreous humor, aqueous humor, mucous, tissue, etc. In some embodiments of the invention, cells such as primary immune cells, such as but not limited to T-cells, may be obtained from a biological sample, such as a blood sample obtained from a subject.

In some embodiments, a scChIA-Drop method of the invention is performed on invertebrate cells, including but not limited to Drosophila cells. In other embodiments, a scChIA-Drop method of the invention is performed on vertebrate cells. In some embodiments, a method of the invention is carried out on mammalian cells, including but not limited to cells from cell lines, primary immune cells (e.g., T-cells), stem cells, diseased cells, healthy cells, etc. In some embodiments, scChIA-Drop is performed on mixed human cells and cells of another organism, non-limiting examples of which are non-human primate cells, mouse cells, etc.

Some embodiments of methods of the invention comprise scChIA-Drop analysis of a. plurality of human cells. Non-limiting examples of mammalian cells that may be used in methods of the invention are cells obtained directly from a subject, cells obtained from a mammalian cell line, cultured mammalian cells, transgenic mammalian cells, etc. In some embodiments of scChIA-Drop methods of the invention are applied to hybrid mammalian stem cells and haplotype-specific multiplex chromatin contact data is generated. The data may be used to assess and determine in allelic-specific genetic interactions.

Cells used in certain methods of the invention, may be obtained from a living animal, e.g., a mammal, or may be obtained from a collection of isolated cells. An isolated cell may be a primary cell, such as those recently isolated from an animal (e.g., cells that have undergone none or only a few population doublings and/or passages following isolation), or may be cells of a cell line that is capable of prolonged proliferation in culture (e.g., for longer than 3 months) or indefinite proliferation in culture (immortalized cells). In some embodiments of the invention, a cell is a somatic cell. Somatic cells may be obtained from an individual, e.g., a human, and cultured according to standard cell culture protocols known to those of ordinary skill in the art. Cells may be obtained from surgical specimens, tissue or cell biopsies, etc, Cells may be obtained from any organ or tissue of interest, including but not limited to: skin, lung, cartilage, brain, CNS, PNS, breast, blood, blood vessel (e.g., artery or vein), fat, pancreas, liver, muscle, gastrointestinal tract, heart, bladder, kidney, urethra, and prostate gland.

In some embodiments, a cell used in conjunction with the invention is a healthy normal cell, which is not known to have a disease, disorder, or abnormal condition. In some embodiments, a cell used in conjunction with methods of the invention is an abnormal cell, for example, a cell obtained from a subject diagnosed as having a disorder, disease, or condition, including, but not limited to a degenerative cell, a neurological disease-bearing cell, a cell model of a disease or condition, an injured cell, etc. In some embodiments of the invention, a cell is an abnormal cell obtained from cell culture, a cell line known to include a disorder, disease, or condition. In some embodiments of the invention, a cell is a control cell. In sonic aspects of the invention a cell can be a model cell for a disease or condition.

Non-limiting examples of a cell that may be used in an embodiment of a method of the invention are one or more of: eukaryotic cells, vertebrate cells, which in some embodiments of the invention may be mammalian cells. A non-limiting example of cells that may be used in methods of the invention are: vertebrate cells, invertebrate cells, and non-human primate cells. Additional, non-limiting examples of cells that may be used in an embodiment of a method of the invention are one or more of: rodent cells, dog cells, cat cells, avian cells, fish cells, cells obtained from a wild animal, cells obtained from a domesticated animal, and other suitable cell of interest. A cell that may be used in certain embodiments of the invention is a human cell. In sonic embodiments a cell is a stein cell, an embryonic stem cell, or embryonic stem cell-like cell. In some embodiments of the invention a cell is a naturally occurring cell and in certain embodiments of the invention a cell is an engineered cell.

Cells useful in embodiments of methods of the invention may be maintained in cell culture following their isolation. Cells may be genetically modified or not genetically modified in various embodiments of the invention. Cells may be obtained from normal or diseased tissue. In some embodiments, cells are obtained from a donor, and their state or type is modified ex vivo using a method of the invention. in certain embodiments of the invention a cell may be a free cell in culture, a free cell obtained from a subject, a cell obtained in a solid biopsy from a subject, organ, or solid culture, etc.

A population or plurality of isolated cells in any embodiment of the invention may be composed mainly or essentially entirely of a particular cell type or of cells in a particular state. In some embodiments, an isolated population or plurality of cells consists of at least 30%, 40%, 50%, 60%, 70%, 80%, 90%, 95%, 96%, 97%, 98%, 99%, or 100% cells of a particular type or state (i.e., the population is at least 30%, 40%, 50%, 60%, 70%, 80%, 90%, 95%, 96%, 97%, 98%, 99%, or 100% pure), e.g., as determined by expression of one or more markers or any other suitable method.

### EXAMPLES

There is a critical need for novel 3D genome mapping technologies to examine multifaceted complex chromatin interactions in single cell with single-molecule precision, Growing evidence supports extensive genome structural stochasticity and transcriptional heterogeneity across phenotypically identical cells, confounding interpretation of 3D genome organization and function. Furthermore, in addition to many protein factors, many non-coding RNAs (ncRNAs) appear to be chromatin-associated, which has led to speculation that many ncRNAs may play essential roles in association with protein factors in chromosomal folding and chromatin remodeling [Rinn J, & Guttman M. Science. (2014), 345(6202):1240-1]. These important biological questions are unlikely to he resolved using current technologies, and there is a critical need for new single-molecule approaches to map multiplex chromatin interactions at single cell level, Methods have now been developed and tested and the results obtained reveal that the majority of chromatin interaction structures consist of multiplex chromatin interactions and many of them are associated with protein and RNA factors.

### Example 1

### Single-Cell ChIA-Drop (scChIA-Drop) Methods and Protocol Development

scChIA-Drop is a de novo, robust single-cell and single-molecule 3D genomic assay using droplet-based microfluidic approaches for single-cell chromatin interaction analysis, scChIA-Drop methods have been used to incorporate two levels of DNA indexing to barcode chromatin fragments in a nucleus-specific and molecule-specific manner.

### Methods

scChIA-Drop Process

scChIA-Drop methods comprise three major elements: single-cell or single-nucleus encapsulation (scChIA-Drop uses isolated nuclei, but may be referred to herein as “single-cell” for convenience), single-cell chromatin barcoding, and single-molecule chromatin barcoding (FIG. 2A-C). scChIA-Drop methods use microfluidics and hydrogel beads to perform two levels of DNA indexing. The hydrogel beads with DNA oligos provide a total barcode capacity of four million (4×10⁶) indexes to achieve molecule-specific indexing in the analysis of multiplex chromatin interactions with single-molecule precision [Zheng M. et al., Nature 566, 558 (2019)].

scChIA-Drop with Cultured Drosphila Cells

### (1) Single-Cell Encapsulation

Initial scChIA-Drop protocols use Drosophila S2 cells, in part due to the small genome size of D. melanogaster. Single-cell encapsulation protocols are adapted for suspension nuclei. One million (10⁶) 52 cells are harvested from cell culture and crosslinked with formaldehyde as previously described [Rao S. S. et al., Cell 159, 1665-80 (2014); Zheng M. et al., Nature 566, 558 (2019)]. The crosslinked S2 cells are then lysed and nuclei are isolated [Rao S. S. et al., Cell 159, 1665-80 (2014); Zheng M. et al., Nature 566, 558 (2019)]. Single-nucleus encapsulation is then performed using a Nadia Innovate microfluidics device (Dolomite Bio, Royston, UK) and a microgel polymer to generate single-nucleus capsules. In some studies, molten agarose is used as the microgel polymer. The encapsulated nuclei are permeabilized and then incubated with reaction mix for in situ chromatin digestion either by HindIII to create sticky DNA ends for later DNA linker ligation or by Tn5 transposase carrying adapter DNA oligos for later DNA barcoding, after which the chromatin fragments in each nucleus in each gel capsule are ready for single-cell (single-nucleus) barcoding.

### (2) Single-Nucleus Chromatin Indexing

The single-cell capsules are then combined with hydrogel beads and a reaction mix containing enzyme to form gel bead in emulsion (GEM) droplets comprising one single-cell capsule and one hydrogel bead per droplet. Hydrogel beads (10X Genomics, Pleasanton, Calif.) are used for nucleus-specific chromatin barcoding with modifications. One set of hydrogel beads comprises four million (4×10⁶) bead-specific oligo barcodes with common features of DNA linker structure [Zheng M. et al., Nature 566, 558 (2019)]. The DNA linker is modified with a HindIII sticky 3′ end, which is compatible with the ends of chromatin fragments digested by HindIII. Once a single-cell droplet is partitioned via microfluidics, the hydrogel bead dissolves, releasing DNA linker barcodes to be enzymatically annealed and ligated to the chromatin fragments in each single-cell droplet, thereby indexing all chromatin fragments in the same nucleus with the same barcode. The droplets are then dissociated, releasing the nuclear-barcoded chromatin material.

### (3) Single-Molecule Chromatin Indexing

The mix of nuclear-indexed chromatin DNA complexes with different nuclear origins is partitioned via microfluidics for single-molecule chromatin indexing with a second set of hydrogel beads and reaction mix, including enzyme, to form GEM droplets comprising a single molecule of chromatin DNA complex and one hydrogel-bead per droplet, as previously described [Zheng M. et al., Nature 566, 558 (2019)]. To create an scChIA-Drop library, the droplets are isothermally incubated to amplify the dual-indexed chromatin DNA fragments. The final scChIA-Drop library is sequenced and analyzed [Zheng M. et al, Nature 566, 558 (2019)].

### Sequencing

A prepared scChIA-Drop library is sequenced using standard sequencing means, which in some instances comprises use of Illumina sequencing method (Illumina., San Diego, Calif.). The scChIA-Drop reads contain both a nucleus-specific barcode and a molecule-specific barcode, thereby achieving single-cell ChIA-Drop analysis with single-molecule precision. Because one set of hydrogel beads comprises millions (10⁶) of bead-specific unique barcodes, the random combinations of the nuclear-specific and molecule-specific indexing steps generate an indexing capacity of trillions (10¹²), which is sufficient to provide unique barcoding to all chromatin molecules in this scChIA-Drop experimental protocol.

### Data Processing

Sequencing data scChIA-Drop library data is processed. In some instances the scChIA-Drop library data is processed using the ChIA-DropBox pipeline [Tian S. Z. et al., bioRxiv January 1:613034 (2019)]. The nuclear barcodes and molecule indexes are used to deconvolute the nuclear origins of chromatin DNA complexes. The MIA-Sig algorithm [Kim M. et al., bioRxiv January 1:665232 (2019)] is further used to de-noise the data and call significant multiplex chromatin contacts.

### Results

Comprehensive single-molecule chromatin interaction data is obtained from at least tens of thousands of nuclei in each scChIA-Drop experiment and is thoroughly analyzed for both single-cell specificity and heterogeneity between cells. The comprehensive single-cell data is also an ensemble profile of multiplex chromatin interactions derived from the million cell population in a scChIA-Drop experiment, and is compared with S2 Hi-C data [Ramirez et al, Mol. Cell 60, 146-162 (2015)], and S2 bulk cell ChIA-Drop data [Zheng M. et al., Nature 566, 558 (2019)] for technical validation and new discoveries.

### Example 2

### scChIA-Drop for Mammalian Cells

Additional optimization and efficiency improvements are included in scChIA-Drop methods and for use with mammalian cells. These protocol adjustments are suitable for use to assess genomes of various organism, including mammals.

### Methods

Experiments are performed using methods scChIA-Drop library preparation, sequencing, and data analysis as described Example 1.

### Mammalian Cells

Human GM12878 cells are used for initial testing of scChIA-Drop methods with human cells, and mouse F1 hybrid mESC F121 (129S1 x CAST) cells are used for initial testing of scChIA-Drop methods with mouse cells. scChIA-Drop experiments are also performed with mixed human and mouse cells as a technical control assessment.

### ChIP-Enrichment

CTCF is the main chromatin architecture protein and RNAPII involves in most gene transcription, therefore, including CTCF and RNAPII enrichment in chromatin interaction analysis enhances detection of most of the chromatin architecture features and related to transcription regulation. To overcome potential issues with noise in scChIA-Drop data due to the large size and complex structure of mammalian genomes, and to enhance the detection of chromatin architecture and transcription regulation features [Tang Z., et al., Cell (2015) 163, 1611-27; Zheng M., et al., Nature (2019) February 566, 558], scChIA-Drop T-cell libraries are ChIP-enriched for CTCF and RNAPII prior to sequencing. Studies are performed including ChIP-enrichment for specific target protein factors such as CTCF and RNAPII in a scChIA-Drop method. Experiments are carried out in mammalian and non-mammalian cells.

### Methods

CTCF-enriched scChIA-Drop methods are performed and the CTCF-enriched scChIA-Drop methods comprise a dual-indexing strategy (nucleus-specific and chromatin-specific as described elsewhere herein), in which individual nuclei of a plurality of cells are barcoded, and the nuclear-indexed chromatin samples partitioned for droplet-specific barcoding in ChIA-Drop library preparation and analysis.

RNAPII-enriched scChIA-Drop methods are performed and the RNAPII-enriched scChIA-Drop methods comprise a dual-indexing strategy (nucleus-specific and chromatin-specific as described elsewhere herein), in which individual nuclei of a plurality of cells are barcoded, and the nuclear-indexed chromatin samples partitioned for droplet-specific barcoding in ChIA-Drop library preparation and analysis.

CTCF-enriched and RNAPII-enriched procedures are performed on mammalian cells and in some studies, a plurality of human cells is encapsulated. In some instances a scChIA-Drop library is prepared using scChIA-Drop methods comprising CTCF-enrichment methods. In some instances a scChIA-Drop library is prepared using scChIA-Drop methods comprising RNAPII-enrichment methods.

### Results

The scChIA-Drop data from human GM12878 cells are compared with ChIA-Drop data from bulk GM12878 cells. The scChIA-Drop data from human GM12878 cells are also compared with scHi-C data available in GM12878 cells [Ramani V. et al., Nat. Methods 14, 263-6 (2017); Tan L. et al., Science 361, 924-8 (2018)] for technical validation and to uncover, for the first time, multiplex chromatin interactions in large numbers of single cells that were not attainable by scHi-C.

Mouse scChIA-Drop data is compared with the available scHi-C data from the same cells for technical validations and discovery of new characteristics in mouse single-cell specificity and heterogeneity in chromatin folding [Nagano et at, Nature 547, 61-67 (2017)]. A major advantage of using hybrid mouse line F121 is its high density of heterozygous SNPs and indels. The comprehensive scChIA-Drop data derived from this cell line provides an unprecedented opportunity to uncover haplotype-specificity of multiplex chromatin interactions genome-wide in single cells and the ensemble property in cell populations. scChIA-Drop experiments are also performed with mixed human and mouse cells as a technical control assessment and results are used to evaluate the scChIA-Drop protocol.

Results indicate the scChIA-Drop protocol is successful for identifying and assessing chromatin interactions at the single-cell level. Results of CTCF-enriched and RNAPII-enriched scChIA-Drop experiments enhance detection of chromatin architecture features related to transcription regulation.

### Example 3

### Single Cell ChIP-Drop (ChIA-Drop) Methods with Primary Human Cells

scChIA-Drop analysis of primary human T-cells isolated from individual blood donors offers a demonstration of the potential of scChIA-Drop methods. The hematopoietic lineage represents an attractive system in which to assess cellular response and differentiation, and provides an excellent opportunity for discovery of 3D genome dynamics and regulatory functions. In addition, immune cells are involved, directly or indirectly, in many diseases such as infections, cancer, autoimmunity and chronic inflammatory conditions. Among immune cells, T-cells have a high level of complexity due to the variations in their differentiated states and functional heterogeneity, which is set by their epigenetic and transcriptional programs, scChIA-Drop methods are used to increase understanding of chromatin interactions in human T-cell subsets and provide necessary genome-level knowledge to enable fine-tuning of cellular responses in many human disease states.

### Methods

scChIA-Drop library preparation, sequencing, and data analysis methods from Examples 1 and 2 are used.

### Isolation and Stimulation of Human Primary T-Cells

Various primary subtypes of blood cells have been isolated, including naive and activated T-cells. Purified. CD4⁺ and CD8⁺ T-cells from human blood are further sorted for naive T-cells (CD45RO-CCR7⁺). Naive T-cells are activated in vitro through their T-cell receptor for various time points. In addition, naive T-cells are differentiated into distinct functional effector subsets (Th0, Th1, Th2, Th17).

Briefly, purified naive T-cells are seeded in 96-well plates and stimulated using anti-CD3/CD28-coated beads (Invitrogen, Waltham, Mass.) under the following T-cell-polarizing conditions: Th0 non-polarizing, anti-IFNg neutralizing antibody+anti-IL-4 neutralizing antibodies; Th1 polarization, neutralizing anti-IL-4 antibody+IL-12; TH2 polarization, anti-IFNg neutralizing antibody+IL-4; Th17 polarization, IL-1-beta, TGFb and IL-23. In addition, cytotoxic effector cells are generated from naive CD8⁺ T-cells with IL-15. The cells population is expanded for two weeks in IL-2-containing media. Cells from the expanded population are used in scChIA-Drop methods and a scChIA-Drop library is prepared using methods Described in Examples 1, 2 and 5 and elsewhere herein.

### ChIP-Enrichment

To overcome potential issues with noise in scChIA-Drop data due to the large size and complex structure of mammalian genomes, and to enhance the detection of chromatin architecture and transcription regulation features, scChIA-Drop T-cell libraries are ChIP-enriched for CTCF and RNAPII prior to sequencing.

CTCF is the main chromatin architecture protein and RNAPII involves in most gene transcription, therefore, including CTCF and RNAPII enrichment in chromatin interaction analysis enhances detection of most of the chromatin architecture features and related to transcription regulation [Tang Z, et al., Cell. 2015:163(7):1611-27; Zheng M, et la., Nature 2019 February, 566(7745):558]. To overcome potential issues with noise in scChIA-Drop data due to the large size and complex structure of mammalian genomes, and to enhance the detection of chromatin architecture and transcription regulation features, scChIA-Drop T-cell libraries are ChIP-enriched for CTCF and RNAPII prior to sequencing. Studies are performed including ChIP-enrichment for specific target protein factors such as CTCF and RNAPII in a scChIA-Drop method. Experiments are carried out in mammalian and non-mammalian cells.

CTCF-enriched scChIA-Drop methods are performed and the CTCF-enriched scChIA-Drop methods comprise a dual-indexing strategy (nucleus-specific and chromatin-specific as described elsewhere herein), in which individual nuclei of a plurality of cells are barcoded, and the nuclear-indexed chromatin samples partitioned for droplet-specific barcoding in ChIA-Drop library preparation and analysis.

RNAPII-enriched scChIA-Drop methods are performed and the RNAPII-enriched scChIA-Drop methods comprise a dual-indexing strategy (nucleus-specific and chromatin-specific as described elsewhere herein), in which individual nuclei of a plurality of cells are barcoded, and the nuclear-indexed chromatin samples partitioned for droplet-specific barcoding in ChIA-Drop library preparation and analysis.

CTCF-enriched and RNAPIII-enriched procedures are performed on mammalian cells and in some studies, a plurality of human cells is encapsulated. In some instances a scChIA-Drop library is prepared using scChIA-Drop methods comprising CTCF-enrichment methods. In some instances a scChIA-Drop library is prepared using scChIA-Drop methods comprising RNAPII-enrichment methods.

### Results

scChIA-Drop data is generated from these T-cell samples and multiplex chromatin interactions are identified and the generated datasets are compared to investigate the dynamics of chromatin topology changes during T-cell activation and differentiation. For technical validation, scChIA-Drop data is also generated from mixed T-cells and is compared with the data obtained from sorted subtype cells to evaluate the single cell-specificity of scChIA-Drop experiments. scChIA-Drop data from T-cell samples is also compared with Hi-C and ChIA-PET data generated from the same T-cell samples and the new methods are validated and data is analyzed to uncover novel insights in chromatin biology. scChIA-Drop data for T-cell samples is verified and integrative analysis is performed with all available data for comprehensive characterization of epigenomic and functional features in T-cells during activation and differentiation. Results indicate the scChIA-Drop protocol is successful for identifying and assessing chromatin interactions at the single-cell level. Results of CTCF-enriched and RNAPII-enriched scChIA-Drop experiments enhance detection of chromatin architecture features related to transcription regulation.

### Example 4

### Transposase Barcoding

The hydrogel bead-based approach is robust and effective for single-cell barcoding in DNA and RNA analysis applications, those applications usually only involve one-step indexing. The scChIA-Drop method as disclosed herein utilizes two barcoding steps (nucleus-specific and molecule-specific) and involves three stages of microfluidic droplet making (FIG. 2A-C). Transposase-based indexing approaches [Vitak S. et al., Nat. Methods 2017 Vol. 14, 302-308] are an efficient alternative.

### Methods

### Transposase-Based Nucleus-Specific Chromatin Indexing

In this approach, encapsulated nuclei are subjected to transposase-based digestion to insert barcode adapters to the chromatin fragments. Two panels of Tn5 transposase (with i5 and i7 adapters, respectively) are incorporated with two sets of 384 unique DNA barcodes, thus yielding a combinatorial barcoding capacity of 150,000 (384×384=147,456).

For the first indexing step, 100,000 encapsulated single nuclei are evenly split into 384 wells (each well contains about 260 single nucleus capsules) for chromatin digestion and barcode insertion by the transposase carrying the i7 oligos with 384 unique barcodes in each of the 384 wells, respectively. Next, in the second indexing step, the i7-barcoded nucleus capsules from the 384 wells are pooled into a single tube, and then divided into 384 wells for the second chromatin digestion and barcode insertion by i5 transposase with unique barcodes in each of the 384 wells, respectively. This two-step of split-and-pool generates unique combinations of dual indexing on chromatin fragments for most of the 100,000 nuclei. The dual-barcoded nuclear capsules are dissolved, and the released mix of chromatin DNA complexes are subjected to molecule-specific chromatin barcoding with the hydrogel beads for scChIA-Drop library construction and subsequent sequencing and analysis (FIG. 1).

scChIA-Drop Methods

scChIA-Drop library preparation, sequencing, and data analysis methods from Examples 1-3 are used.

### Results

Although the hydrogel bead-based approach (Examples 1-3) has at least a 20× larger nucleus indexing capacity than the transposase-based approach (4,000,000 vs. 150,000), the transposase-based approach simplifies the overall scChIA-Drop procedure because the chromatin fragmentation and barcoding are done in one step. In contrast, chromatin fragmentation, nuclei barcoding, and the molecule barcoding are separate steps in the hydrogel bead-based strategy (Examples 1-3).

### Example 5

### Enriched Single-Cell ChIA-Drop

Studies are performed including ChIP-enrichment for specific target protein factors such as CTCF and RNAPII in a scChIA-Drop method. Experiments are carried out in mammalian and non-mammalian cells. CTCF is the main chromatin architecture protein and RNAPII involves in most gene transcription, therefore, including CTCF and RNAPII enrichment in chromatin interaction analysis enhances detection of most of the chromatin architecture features and related to transcription regulation [Tang Z, et al., Cell. 2015:163(7):1611-27; Zheng M, et la., Nature 2019 February, 566(7745):558].

### Methods

CTCF-enriched scChIA-Drop methods are performed and the CTCF-enriched scChIA-Drop methods comprise a dual-indexing strategy (nucleus-specific and chromatin-specific as described elsewhere herein), in which individual nuclei of a plurality of cells are barcoded, and the nuclear-indexed chromatin samples partitioned for droplet-specific barcoding in ChIA-Drop library preparation arid analysis.

RNAPIII-enriched scChIA-Drop methods are performed and the RNAPII-enriched scChIA-Drop methods comprise a dual-indexing strategy (nucleus-specific and chromatin-specific as described elsewhere herein), in which individual nuclei of a plurality of cells are barcoded, and the nuclear-indexed chromatin samples partitioned for droplet-specific barcoding in ChIA-Drop library preparation and analysis.

CTCF-enriched and RNAPII-enriched procedures are performed on mammalian cells, and in some studies, a plurality of human cells is encapsulated and a scChIA-Drop library is prepared.

### Results

Results of CTCF-enriched and RNAPII-enriched scChIA-Drop experiments enhance detection of chromatin architecture features related to transcription regulation.

### Equivalents

Although several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e, elements that are conjunctively present in some cases and disjunctively present in other cases. Other elements may optionally be present other than the elements specifically identified by the “and/or” clause, whether related or unrelated to those elements specifically identified, unless clearly indicated to the contrary.

All references, patents and patent applications and publications that are cited or referred to in this application are incorporated by reference in their entirety herein.

What is claimed is:

1. A method of single-cell and single-molecule chromatin DNA barcoding, comprising:
(a) preparing a plurality of encapsulated single cell beads;
(b) generating single-cell barcoded chromatin DNA in the prepared encapsulated single cells; and
(c) performing single-molecule chromatin DNA barcoding on the generated single-cell barcoded chromatin DNA, wherein the resulting barcoded chromatin DNA complex is single-cell/single-molecule barcoded chromatin DNA complex.

2. The method of claim 1, further comprising:
(d) amplifying the single-cell/single-molecule barcoded chromatin DNA complex;
(e) generating sequencing data from the amplified DNA sequences; and
(f) analyzing one or more of the generated sequencing data and the amplified DNA sequences.

3. The method of claim 1, wherein a means for preparing the plurality of encapsulated single-cell beads comprises mixing a microgel polymer and a single-cell suspension.

4. The method of claim 1, wherein a means of generating single-cell barcoded chromatin DNA complex in the prepared encapsulated single-cell beads, comprises one or more of:
(a) lysing the cells in the encapsulated single-cell beads;
(b) digesting chromatin in cell nuclei in the encapsulated single-cell beads into chromatin fragments; and
(c) generating single-cell droplets by combining the encapsulated single-cell beads comprising the chromatin fragments with:
(i) a plurality of a second gel bead comprising an independently selected single-cell-indexed DNA linker comprising a barcode indexed to the single cell; and
(ii) a first reaction mix comprising a first independently selected enzyme;
wherein: the generated single-cell droplets each comprises one of the single-cell beads and one of the second gel beads; the second gel bead dissolves releasing the single-cell-indexed DNA linkers, and the released single-cell-indexed linkers are attached to the chromatin fragments forming single-cell-indexed barcoded chromatin DNA complexes.

5. The method of claim 1, wherein a means of performing single-molecule chromatin DNA barcoding on the generated single-cell barcoded chromatin DNA, comprises: one or more of:
(a) pooling the cell nuclei and releasing the single-cell-indexed barcoded chromatin DNA complexes from the pooled nuclei;
(b) generating a plurality of chromatin droplets by combining the released single-cell-indexed barcoded chromatin DNA complexes with:
(i) a plurality of a third-gel bead comprising independently selected single-molecule-indexed DNA linkers comprising a plurality of barcodes indexed for single-molecule barcoding and
(ii) a second reaction mix comprising a second independently selected enzyme;
wherein the generated chromatin droplets comprise the single-cell-indexed barcoded chromatin DNA complexes and one of the third-gel beads; the third-gel bead dissolves releasing the single-molecule-indexed DNA linkers; and the released single-molecule-indexed linkers are attached to an end of the chromatin fragments in the single-cell indexed barcoded chromatin DNA complexes forming chromatin DNA complexes comprising a single-cell indexed barcode and a single-molecule-indexed barcode.

6. The method of claim 4, wherein a means for digesting the chromatin comprises a restriction enzyme digestion.

7. The method of claim 6, wherein the restriction enzyme digestion creates sticky DNA ends.

8. The method of claim 6, wherein the restriction enzyme is a 4-bp cutter or a 6-bp cutter, wherein optionally the 4-bp cutter is MboI and optionally the 6-bp cutter is HindIII.

9. The method of claim 4, wherein digesting the chromatin results in DNA fragments of 300-6000 bp.

10. The method of claim 4, wherein a means for digesting the chromatin comprises a transposase digestion.

11. The method of claim 10, wherein the transposase comprises a Tn5 transposase polypeptide.

12. The method of claim 11, wherein the transposase polypeptide is carrying an adapter DNA oligonucleotide for barcoding.

13. The method of claim 5, wherein prior to combining the chromatin fragments with the plurality of single-cell-indexed barcoded linkers, the population of chromatin fragments is adjusted in solution to a solution concentration of 0.5 ng DNA/μl.

14. The method of claim 5, wherein prior to combining the chromatin fragments with the plurality of single-cell-indexed barcoded linkers the population of chromatin DNA complexes is enriched for a chromatin protein.

15. The method of claim 14, wherein the enrichment comprises incubating the population of chromatin fragments with a monoclonal antibody specific for the chromatin protein to form chromatin DNA complexes bound to the monoclonal antibody, isolating the chromatin DNA complexes bound to the monoclonal antibody, and removing the monoclonal antibody to form a population of chromatin DNA complexes each complex comprising the chromatin protein.

16. The method of claim 14 or 15, wherein the chromatin protein is RNAPII, RARA ER, or CTCF.

17. The method of any one of claims 1-15, wherein the gel beads comprise gel beads in emulsion (GEMs).

18. The method of claim 17, wherein each GEM contains multiple copies of a DNA construct comprising a PCR priming site, a sequence reading site, one or both of a single-cell indexed barcode and a single molecule-indexed barcode, and a random priming nucleotide sequence.

19. The method of claim 18, wherein the random priming nucleotide sequence is a random 8-mer.

20. The method of claim 4, wherein one or both of the single-cell-indexed barcode and the single-molecule-indexed barcode comprises ten or more nucleotides.

21. The method of claim 4, wherein one or both of the single-cell-indexed barcode and the single-molecule-indexed barcode comprises 8, 9, 10, 11, 12, or more nucleotides.

22. The method of claim 4, wherein one or both of the single-cell-indexed barcode and the single-molecule-indexed barcode comprises a 15 nt to 25 nt barcode or a 16 nt to 20 nt barcode.

23. The method of claim 1, wherein the chromatin DNA complexes comprise chromatin DNA and chromatin protein.

24. The method of claim 5, wherein a means of releasing the barcoded chromatin DNA complexes in (a) comprises lysing the pooled nuclei.

25. The method of claim 4, wherein the chromatin is released from the cell nuclei before digesting the chromatin into chromatin DNA fragments.

26. The method of claim 25, wherein a means for releasing the chromatin from the cell nuclei comprises one or more of: crosslinking the nucleus with a crosslinking reagent, permeabilizing the crosslinked nucleus with a permeabilizing reagent, and digesting the permeabilized nucleus.

27. The method of claim 4, wherein a means for lysing the single cell in the encapsulated single-cell bead comprises:
(a) crosslinking the single cell with a crosslinking reagent to form a crosslinked single cell comprising a crosslinked nucleus,
(b) lysing the crosslinked single cell,
(c) isolating the crosslinked cell nucleus from the lysed single cell, and
(d) permeabilizing the isolated crosslinked cell nucleus with a permeabilizing reagent.

28. The method of claim 26 or 27, wherein the crosslinking reagent comprises formaldehyde.

29. The method of claim 28, wherein the formaldehyde is 1% (w/v) formaldehyde.

30. The method of claim 26, wherein the permeabilizing reagent comprises Sodium Dodecyl Sulphate (SDS).

31. The method of claim 30, wherein the SDS is 0.5% SDS.

32. The method of claim 26, wherein the cross-linked permeabilized cell nucleus is fragmented by sonication prior to digestion.

33. The method of claim 2, wherein a means of the amplifying the barcoded chromatin DNA comprises isothermal incubation of the indexed single-cell and single-molecule barcoded chromatin DNA at about 30° C. for about 8-16 hours.

34. The method of claim 33, wherein one or both of the amplified indexed single-cell and single-molecule barcoded chromatin DNA fragments are subjected to one or more of end repair, A-tailing, and adapter ligation prior to sequencing.

35. The method of claim 2, wherein the sequencing is 150 by sequencing.

36. The method of claim 4, wherein the digesting step is performed using a restriction enzyme digestion.

37. The method of claim 1, further comprising determining a chromatin DNA interaction in the single cell at a single-molecule level.

38. A method of single-cell and single-molecule chromatin DNA barcoding, comprising:
(a) mixing a microgel polymer and a single cell/nuclei suspension to create a plurality of encapsulated single-cell beads;
(b) lysing the cells in the encapsulated single cell beads;
(c) digesting chromatin in the cell nuclei in the encapsulated single-cell beads into chromatin fragments;
(d) generating single-cell droplets by combining the encapsulated single-cell beads comprising the chromatin fragments with:
(i) a plurality of a second gel bead comprising an independently selected single-cell-indexed DNA linker comprising a barcode indexed to the single cell; and
(ii) a first reaction mix comprising a first independently selected enzyme;
wherein: the generated single-cell droplets each comprises one of the single-cell beads and one of the second gel beads; the second gel bead dissolves releasing the single-cell-indexed DNA linkers, and the released single-cell-indexed linkers are attached to the chromatin fragments forming single-cell-indexed barcoded chromatin DNA complexes;
(e) pooling the cell nuclei and releasing the single-cell-indexed barcoded chromatin DNA complexes from the pooled nuclei;
(f) generating a plurality of chromatin droplets by combining the released single-cell-indexed barcoded chromatin DNA complexes with:
(iii) a plurality of a third-gel bead comprising independently selected single-molecule-indexed DNA linkers comprising a plurality of barcodes indexed for single-molecule barcoding and
(iv) a second reaction mix comprising a second independently selected enzyme;
wherein the generated chromatin droplets comprise the single-cell-indexed barcoded chromatin DNA complexes and one of the third-gel beads; the third-gel bead dissolves releasing the single-molecule-indexed DNA linkers; and the released single-molecule-indexed linkers are attached to an end of the chromatin fragments in the single-cell indexed barcoded chromatin DNA complexes forming chromatin DNA complexes comprising a single-cell indexed barcode and a single-molecule-indexed barcode;
(g) amplifying the barcoded chromatin DNA;
(h) generating sequencing data from the amplified DNA sequences; and
(i) analyzing one or more of the generated sequencing data and the amplified DNA sequences.

39. The method of claim 38, wherein a means for digesting the chromatin in step (c) comprises a restriction enzyme digestion.

40. The method of claim 39, wherein the restriction enzyme digestion creates sticky DNA ends.

41. The method of claim 39, wherein the restriction enzyme is a 4-bp cutter or a 6-bp cutter, wherein optionally the 4-bp cutter is MboI and optionally the 6-bp cutter is HindIII.

42. The method of claim 38, wherein digesting the chromatin results in DNA fragments of 300-6000 bp.

43. The method of claim 38, wherein a means for digesting the chromatin in step (c) comprises a transposase digestion.

44. The method of claim 43, wherein the transposase comprises Tn5 transposase polypeptide.

45. The method of claim 44, wherein the transposase polypeptide is carrying an adapter DNA oligonucleotide for barcoding.

46. The method of claim 38, wherein prior to combining the chromatin fragments with the plurality of single-cell-indexed barcoded linkers, the population of chromatin fragments is adjusted in solution to a solution concentration of 0.5 ng DNA/μl.

47. The method of claim 38, wherein prior to combining the chromatin fragments with the plurality of single-cell-indexed barcoded linkers the population of chromatin DNA complexes is enriched for a chromatin protein.

48. The method of claim 47, wherein the enrichment comprises incubating the population of chromatin fragments with a monoclonal antibody specific for the chromatin protein to form chromatin DNA complexes bound to the monoclonal antibody, isolating the chromatin DNA complexes bound to the monoclonal antibody, and removing the monoclonal antibody to form a population of chromatin DNA complexes each complex comprising the chromatin protein.

49. The method of claim 47 or 48, wherein the chromatin protein is RNAPII, RARA, ER, or CTCF.

50. The method of claim 38, wherein the gel beads comprise gel beads in emulsion (GEMs).

51. The method of claim 50, wherein each GEM contains multiple copies of a DNA construct comprising a PCR priming site, a sequence reading site, one or both of a single-cell indexed barcode and a single molecule-indexed barcode, and a random priming nucleotide sequence.

52. The method of claim 51, wherein the random priming nucleotide sequence is a random 8-mer.

53. The method of claim 38, wherein one or both of the single-cell-indexed barcode and the single-molecule-indexed barcode comprises ten or more nucleotides.

54. The method of claim 38, wherein one or both of the single-cell-indexed barcode and the single-molecule-indexed barcode comprises 8, 9, 10, 11, 12, or more nucleotides.

55. The method of claim 38, wherein one or both of the single-cell-indexed barcode and the single-molecule-indexed barcode comprises a 15 nt to 25 nt barcode or a 16 nt to 20 nt barcode.

56. The method of claim 38, wherein the chromatin DNA complexes comprise chromatin DNA and chromatin protein.

57. The method of claim 38, wherein a means of releasing the barcoded chromatin DNA complexes in (e) comprises lysing the pooled nuclei.

58. The method of claim 38, wherein the chromatin is released from the cell nuclei before digesting the chromatin into chromatin DNA fragments.

59. The method of claim 58, wherein a means for releasing the chromatin from the cell nuclei comprises one or more of: crosslinking the nucleus with a crosslinking reagent, permeabilizing the crosslinked nucleus with a permeabilizing reagent, and digesting the permeabilized nucleus.

60. The method of claim 38, wherein a means for lysing the single cell in the encapsulated single-cell bead comprises:
(a) crosslinking the single cell with a crosslinking reagent to form a crosslinked single cell comprising a crosslinked nucleus,
(b) lysing the crosslinked single cell,
(c) isolating the crosslinked cell nucleus from the lysed single cell, and
(d) permeabilizing the isolated. crosslinked cell nucleus with a permeabilizing reagent.

61. The method of claim 59 or 60, wherein the crosslinking reagent comprises formaldehyde.

62. The method of claim 61, wherein the formaldehyde is 1% (w/v) formaldehyde.

63. The method of claim 59, wherein the permeabilizing reagent comprises Sodium Dodecyl Sulphate (SDS).

64. The method of claim 63, wherein the SDS is 0.5% SDS.

65. The method of claim 59, wherein the cross-linked permeabilized cell nucleus is fragmented by sonication prior to digestion.

66. The method of claim 38, wherein a means of the amplifying the barcoded chromatin DNA comprises isothermal incubation of the indexed single-cell and single-molecule barcoded chromatin DNA at about 30° C. for about 8-16 hours.

67. The method of claim 66, wherein one or both of the amplified indexed single-cell and single-molecule barcoded chromatin DNA fragments are subjected to one or more of end repair, A-tailing, and adapter ligation prior to sequencing.

68. The method of claim 38, wherein the sequencing is 150-bp sequencing.

69. The method of claim 38, wherein the digesting step is performed using a restriction enzyme digestion.

70. The method of claim 38, further comprising determining a chromatin DNA interaction in the single cell at a single-molecule level.

71. A method of mapping chromatin DNA complexes, comprising:
(a) determining the amplified DNA sequences using a method of claim 2 and
(b) analyzing the amplified DNA sequences.

72. A method of mapping chromatin DNA complexes, comprising:
(a) determining the amplified DNA sequences using a method of claim 38, and
(b) analyzing the amplified DNA sequences.

73. The method of claim 71 or 72, wherein a means of analyzing the amplified DNA sequences comprises a ChIA-DropBox pipeline method.

74. A method of ChIA-DropBox pipeline sequence analysis, comprising:
(a) reading the sequence data generated using a method of claims 2;
(b) identifying one or more of the barcodes on the barcoded chromatin DNA based on the reading;
(c) calling of GEMS based on the barcode identification;
(d) identifying significant chromatin DNA complexes; and
(e) visualizing the data obtained in (d).

75. A method of ChIA-DropBox pipeline sequence analysis, comprising:
(a) reading the sequence data generated using a method of claim 38;
(b) identifying one or more of the barcodes on the barcoded chromatin DNA based on the reading;
(c) calling of GEMS based on the barcode identification;
(d) identifying significant chromatin DNA complexes; and
(e) visualizing the data obtained in (d).

76. A method of a single-cell chromatin identification, the method comprising:
(a) preparing a plurality of single-cell gel beads, each comprising a cell nucleus of a single cell, wherein the cell nucleus comprises chromatin DNA complexes;
(b) digesting the chromatin DNA complexes into chromatin DNA fragments;
(c) mixing the single-cell gel beads comprising the chromatin DNA fragments with:
(i) a plurality of a second gel bead, each comprising a plurality of an indexed barcode linker comprising a barcode indexed to the single cell; and
(ii) reagents comprising an enzyme capable of ligating the barcodes to the chromatin DNA fragments,
(d) partitioning the single-cell gel beads and the second gel beads in the mixture into individual single-cell droplets comprising at least one of the single-cell gel beads and at least one of the second gel beads; and
(e) releasing the indexed barcode linkers within each single-cell droplet, wherein the released single-cell indexed barcode linkers add one of the indexed single-cell barcodes to a chromatin DNA fragment in the single-cell droplet, thereby generating indexed single-cell barcoded chromatin DNA fragments, wherein the chromatin DNA from the single cell is identified by the presence of the chromatin DNA fragments comprising the indexed single-cell barcode.

77. The method of claim 76, further comprising determining a chromatin DNA interaction in the single cell at a single-molecule level.

78. The method of claim 77, wherein a means of determining the chromatin DNA interaction at the single molecule level comprises a Chia-PET, Hi-C, or a ChIA-drop method.
